(19) United States
(12) Patent Application Publication
(10) Pub. No.: US 2005/0182090 A1
(43) Pub. Date: Aug. 18, 2005

(54) Pharmaceutical composition comprising a monoamine neurotransmitter re-uptake inhibitor and a dopamine agonist

(75) Inventors: Joachim Mierau, Mainz (DE); Michael P. Pieper, Biberach (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim Pharma GmbH & Co. KG, Ingelheim (DE)

(21) Appl. No.: 11/040,559

(22) Filed: Jan. 21, 2005

(30) Foreign Application Priority Data
Jan. 22, 2004 (EP) 04 001 281
Mar. 11, 2004 (EP) 04 005 817

(51) Int. Cl. A61K 31/46; A61K 31/137

(52) U.S. Cl. 514/304; 514/649

(57) ABSTRACT

The invention relates to a pharmaceutical composition comprising a monoamine neurotransmitter re-uptake inhibitor comprising a 2,3-disubstituted tropane moiety, or a tautomer, a pharmaceutically acceptable salt, solvate, or physiologically functional derivative thereof, and at least one dopamine agonist or a pharmaceutically acceptable salt, solvate, or physiologically functional derivative thereof.

This applications claims priority of EP Application Nos. 04001281 and 04005817, which are incorporated herein by reference in their entirties.

### BACKGROUND OF THE INVENTION

1. Technical Field

The present invention relates to a combination of a monoamine neurotransmitter re-uptake inhibitor and a dopamine agonist, and the use of the combination in treating neurodegenerative conditions such as Alzheimer's Disease.

2. Background Information

Alzheimer's Disease and dementia of Alzheimer-type are poorly understood neurodegenerative conditions mainly affecting the elderly, but also younger people who are generally genetically predisposed to it.

One postulated method of treatment comprises the administration of dopamine agonists which act on the cholinergic system.

However this method suffers from the disadvantages that these compounds induce a range of side-effects including diarrhea, salivation, and nausea.

The tropane derivative having dopamine reuptake inhibitor activity for use according to the invention may, in particular, be tropane derivatives such as those disclosed by patent applications EP 604355, EP 604352, U.S. Pat. No. 5,444,070, EP 604354, WO 95/28401, and WO 97/30997, all of which are encorporated herein in their entirties.

The International patent application WO 97/30997 discloses tropane derivatives, which are monoamine neurotransmitter re-uptake inhibitor. Similar compounds are known from the International patent application WO 93/09814.

However, there is no hint to combine these compounds with a dopamine agonist.

The present invention provides a new and surprisingly effective combination of a dopamine agonist and a monoamine neurotransmitter re-uptake inhibitor for separate, sequential, or simultaneous administration.

Surprisingly, an unexpectedly beneficial therapeutic effect can be observed if dopaminergic agonists are used in combination with a monoamine neurotransmitter re-uptake inhibitor comprising a 2,3-disubstituted tropane moiety:

### BRIEF SUMMARY OF THE INVENTION

Accordingly, the invention relates to a pharmaceutical composition comprising a monoamine neurotransmitter re-uptake inhibitor comprising a 2,3-disubstituted tropane moiety, or a tautomer, a pharmaceutically acceptable salt, solvate, or physiologically functional derivative thereof, and at least one dopamine agonist or a pharmaceutically acceptable salt, solvate, or physiologically functional derivative thereof.

The present invention provides a greater than expected improvement than would be expected from administration of the active ingredients alone in the condition of subjects suffering from a neurodegenerative disorder with an associated cognitive deficit, such as Alzheimer's Disease, dementia of Alzheimer-type, Lewy Body dementia, fronto-temporal dementia, or from a cognitive deficit that may arise from a normal process, such as aging-like cerebrovascular dementia, multi-infarct dementia, and milder forms, such as age-associated memory impairment (AAMI) or mild cognitive impairment (MCI), or from an abnormal process, such as injury, than would be expected from administration of the active ingredients alone. Further, the combination allows for a lower overall dose of each of the active ingredients to be administered, thus reducing side effects and decreasing any reduction in the effectiveness of each of the active ingredients over time.

There is also provided a kit of parts comprising at least two separate unit dosage forms (A) and (B):
(A) comprising a composition a monoamine neurotransmitter re-uptake inhibitor comprising a 2,3-disubstituted tropane moiety, or a tautomer, a pharmaceutically acceptable salt, solvate, or physiologically functional derivative thereof, and optionally a pharmaceutically acceptable carrier;
(B) comprising a composition containing one or more dopamine agonists or a pharmaceutically acceptable salt, solvate, or physiologically functional derivative thereof, and optionally a pharmaceutically acceptable carrier,
   for simultaneous, sequential, or separate administration.

There is also provided the use of a combination of a monoamine neurotransmitter re-uptake inhibitor comprising a 2,3-disubstituted tropane moiety, or a tautomer, a pharmaceutically acceptable salt, solvate, or physiologically functional derivative thereof and at least one dopamine agonist or a pharmaceutically acceptable salt, solvate, or physiologically functional derivative thereof in a combined form, or separately, or separately and sequentially, wherein the sequential administration is close in time or remote in time, for the prevention or treatment of a disease or a disorder, which is responsive to the inhibition of monoamine neurotransmitter re-uptake and or to dopamine agonism.

There is also disclosed a method of prevention or treatment of a disease or disorder, which disease or disorder is responsive to the inhibition of monoamine neurotransmitter re-uptake, which method comprises administration of effective amounts of a monoamine neurotransmitter re-uptake inhibitor comprising a 2,3-disubstituted tropane moiety, or a tautomer, a pharmaceutically acceptable salt, solvate, or physiologically functional derivative thereof, and at least one dopamine agonist or a pharmaceutically acceptable salt, solvate, or physiologically functional derivative thereof to a patient in need thereof in a combined form, or separately, or separately and sequentially wherein the sequential administration is close in time or remote in time.

Diseases and/or disorders that may be prevented or treated by the present invention include: depression, dementia, pseudodementia, presenile dementia, senile dementia, dementia of Alzheimer-type, fronto-temporal dementia, HIV-related dementia, multi-infarct dementia, cerebrovascular dementia, Alzheimer's Disease, Lewy Body disease, Down syndrome, Pick's disease, memory deficits, attention deficits, cognitive dysfunction, memory dysfunction, age associated memory impairment, mild cognitive impairment, ageing-associated cognitive decline, age-related cognitive decline, multiple system atrophy, and neurodegenerative disorder with an associated cognitive deficit.

### DETAILED DESCRIPTION OF THE INVENTION

As a rule the monoamine neurotransmitter re-uptake inhibitor comprising a 2,3-disubstituted tropane moiety are those which are disclosed by International patent applications WO 93/09814 and WO 97/30997.

For use according to the invention, it is preferable to use a compound of the general formula (I) 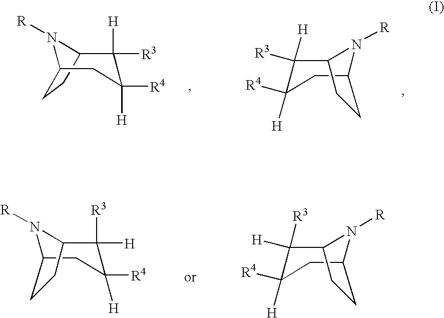
or a pharmaceutical acceptable addition salt thereof, or the N-oxide thereof, wherein
R is hydrogen, alkyl, alkenyl, alkynyl, cycloalkyl, cycloalkylalkyl or 2-hydroxyethyl;
R³ is
   CH₂—X—R′, wherein
      X is O, S, or NR″; wherein
         R″ is hydrogen or alkyl; and
      R′ is
         alkyl, alkenyl, alkynyl, cycloalkyl, cycloalkylalkyl, or —CO-alkyl;
         heteroaryl, which may be substituted one or more times with alkyl, cycloalkyl, or cycloalkylalkyl;
         phenyl, which may be substituted one or more times with substituents selected from the group consisting of halogen, CF₃, CN, alkoxy, alkyl, alkenyl, alkynyl, amino, nitro, and heteroaryl;
         phenylphenyl;
         pyridyl, which may be substituted one or more times with substituents selected from the group consisting of halogen, CF₃, CN, alkoxy, alkyl, alkenyl, alkynyl, amino, nitro, and heteroaryl;
         thienyl, which may be substituted one or more times with substituents selected from the group consisting of halogen, CF₃, CN, alkoxy, alkyl, alkenyl, alkynyl, amino, nitro, and heteroaryl; or
         benzyl, which may be substituted one or more times with substituents selected from the group consisting of halogen, CF₃, CN, alkoxy, alkyl, alkenyl, alkynyl, amino, nitro, and heteroaryl; or
   (CH₂)ₙCO₂R¹¹, COR¹¹, or CH₂R¹², wherein
      R¹¹ is
         alkyl, cycloalkyl, or cycloalkylalkyl;
         phenyl, which may be substituted one or more times with substituents selected from the group consisting of halogen, CF₃, CN, alkoxy, alkyl, alkenyl, alkynyl, amino, nitro, and heteroaryl;
         phenylphenyl;
         pyridyl, which may be substituted one or more times with substituents selected from the group consisting of halogen, CF₃, CN, alkoxy, alkyl, alkenyl, alkynyl, amino, nitro, and heteroaryl;
         thienyl or O-thienyl, which may be substituted one or more times with substituents selected from the group consisting of halogen, CF₃, CN, alkoxy, alkyl, alkenyl, alkynyl, amino, nitro, and heteroaryl; or benzyl;
      n is 0 or 1; and
      R¹² is
         O-phenyl, which may be substituted one or more times with substituents selected from the group consisting of halogen, CF₃, CN, alkoxy, alkyl, alkenyl, alkynyl, amino, nitro, and heteroaryl; or
         O—CO-phenyl, which may be substituted one or more times with substituents selected from the group consisting of halogen, CF₃, CN, alkoxy, alkyl, alkenyl, alkynyl, amino, nitro, and heteroaryl; or
   CH═NOR′, wherein
      R′ is
         hydrogen or O-hydrogen;
         alkyl, O-alkyl, cycloalkyl, cycloalkylalkyl, alkenyl, alkynyl or aryl, all of which may be substituted with —COOH;
         —COO-alkyl;
         —COO-cycloalkyl; or
         phenyl, which may be substituted one or more times with substituents selected from the group consisting of halogen, CF₃, CN, alkyl, cycloalkyl, alkoxy, cycloalkoxy, alkenyl, alkynyl, amino, and nitro;
R⁴ is
   3,4-methylenedioxyphenyl; or
   phenyl, benzyl, naphthyl, or heteroaryl, all of which may be substituted one or more times with substituents selected from the group consisting of halogen, CF₃, CN, alkoxy, cycloalkoxy, alkyl, cycloalkyl, alkenyl, alkynyl, amino, nitro, and heteroaryl.

In a special embodiment of the compound of general formula (I), R³ is
1,2,4-oxadiazol-3-yl, which may by substituted in the 5 position with
   alkyl, cycloalkyl, or cycloalkylalkyl;
   phenyl, which may be substituted one or more times with substituents selected from the group consisting of halogen, CF₃, CN, alkoxy, alkyl, alkenyl, alkynyl, amino, nitro, and heteroaryl; phenylphenyl; or
   benzyl, which may be substituted one or more times with substituents selected from the group consisting of halogen, CF₃, CN, alkoxy, alkyl, alkenyl, alkynyl, amino, nitro, and heteroaryl; or
1,2,4-oxadiazol-5-yl, which may by substituted in the 3 position with
   alkyl, cycloalkyl, or cycloalkylalkyl;
   phenyl, which may be substituted one or more times with substituents selected from the group consisting of halogen, CF₃, CN, alkoxy, alkyl, alkenyl, alkynyl, amino, nitro, and heteroaryl; phenylphenyl;
   benzyl, which may be substituted one or more times with substituents selected from the group consisting of halogen, CF₃, CN, alkoxy, alkyl, alkenyl, alkynyl, amino, nitro, and heteroaryl;
   pyridyl, which may be substituted one or more times with substituents selected from the group consisting of halogen, CF₃, CN, alkoxy, alkyl, alkenyl, alkynyl, amino, nitro and heteroaryl; or
   thienyl, which may be substituted one or more times with substituents selected from the group consisting of halogen, CF₃, CN, alkoxy, alkyl, alkenyl, alkynyl, amino, nitro and heteroaryl.

In a further special embodiment of the compound of general formula (I), R³ is
CH₂—X—R′, wherein
   X is
      O, S, or NR″; wherein
         R″ is hydrogen or alkyl; and
   R′ is alkyl, alkenyl, alkynyl, cycloalkyl, cycloalkylalkyl, or —CO-alkyl.

In a still further embodiment of the compound of general formula (I), R³ is
CH═NOR′, wherein
   R′ is
      hydrogen;
      alkyl, cycloalkyl, cycloalkylalkyl, alkenyl, alkynyl or aryl, all of which may be substituted with —COOH;
      —COO-alkyl;
      —COO-cycloalkyl; or
      phenyl, which may be substituted one or more times with substituents selected from the group consisting of halogen, CF₃, CN, alkyl, cycloalkyl, alkoxy, cycloalkoxy, alkenyl, alkynyl, amino, and nitro.

In a further special embodiment of the compound of general formula (I), R⁴ is phenyl, which is substituted once or twice with substituents selected from the group consisting of halogen, CF₃, CN, alkoxy, cycloalkoxy, alkyl, cycloalkyl, alkenyl, alkynyl, amino, nitro, and heteroaryl.

In a more special embodiment, R⁴ is phenyl substituted once or twice with chlorine.

In a further special embodiment, the tropane derivative having dopamine reuptake inhibitor activity is a (1R,2R,3S)-2,3-disubstituted tropane derivative of formula (I).

In a still further embodiment, the tropane derivative having dopamine reuptake inhibitory activity is a compound of general formula (I), wherein
R³ is
   —CH₂—X—R′, wherein X is O or S, and R′ is methyl, ethyl, propyl, or cyclopropylmethyl;
   —CH═NOR′, wherein R′ is hydrogen or alkyl, or
   1,2,4-oxadiazol-5-yl, which may by substituted in the 3 position with alkyl.

In a still further embodiment, the tropane derivative having dopamine reuptake inhibitory activity is a compound of general formula (I) wherein R is hydrogen, methyl, ethyl or propyl.

In a still further embodiment, the tropane derivative having dopamine reuptake inhibitory activity is a compound of general formula (I) wherein R⁴ is 3,4-dichlorophenyl.

Preferably those monoamine neurotransmitter re-uptake inhibitor comprising a 2,3-disubstituted tropane moiety are compounds of formula (I1) 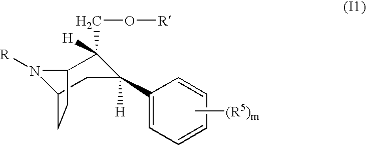
wherein
R represents a hydrogen atom or a C₁₋₆ alkyl group, preferably a hydrogen atom, a methyl or an ethyl group;
R⁵ each independently represents a halogen atom or a CF₃ or cyano group, preferably a fluorine, chlorine or bromine atom;
R represents a hydrogen atom or a C₁₋₆ alkyl or C₃₋₆-cycloalkyl-C₁₋₃-alkyl group, preferably a methyl, ethyl or n-propyl group; and
m is 0 or an integer from 1 to 3, preferably 1 or 2;
   or a tautomer, a pharmaceutically acceptable salt, solvate, or physiologically functional derivative thereof.

As used herein, the expression “C₁₋₆ alkyl” includes methyl and ethyl groups, and straight-chained and branched propyl, butyl, pentyl, and hexyl groups. Particular alkyl groups are methyl, ethyl, n-propyl, isopropyl, and t-butyl.

The expression “C₃₋₆ cycloalkyl,” as used herein, includes cyclic propyl, butyl, pentyl, and hexyl groups, such as cyclopropyl and cyclohexyl.

The term “halogen,” as used herein, includes fluorine, chlorine, bromine, and iodine, of which fluorine and chlorine are preferred.

The term “physiologically functional derivative,” as used herein, includes derivatives obtained from the compound of formula (I) under physiological conditions, these are, for example, N-oxides, which are formed under oxidative conditions.

The term “pharmaceutically acceptable acid addition salt,” as used herein, includes those salts that are selected from among the acid addition salts formed with hydrochloric acid, hydrobromic acid, sulphuric acid, phosphoric acid, methanesulphonic acid, acetic acid, fumaric acid, succinic acid, lactic acid, citric acid, tartaric acid, and maleic acid, the salts obtained from hydrochloric acid, hydrobromic acid, sulphuric acid, phosphoric acid, and acetic acid being particularly preferred. The salts of citric acid are of particular significance.

In a special embodiment, the tropane derivative having dopamine reuptake inhibitor activity is a compound of the general formula (I) selected from:
(1R,2R,3S)-2-(3-Cyclopropyl-1,2,4-oxadiazol-5-yl)-3-(4-fluorophenyl)-tropane;
(1R,2R,3S)-2-(3-Phenyl-1,2,4-oxadiazol-5-yl)-3-(4-fluorophenyl)-tropane;
(1R,2R,3S)-2-(3-Phenyl-1,2,4-oxadiazol-5-yl)-3-(4-methylphenyl)-tropane;
(1R,2R,3S)-2-(3-Benyl-1,2,4-oxadiazol-5-yl)-3-(4-fluorophenyl)-tropane;
(1R,2R,3S)-2-(3-(4-Phenyl-phenyl)-1,2,4-oxadiazol-5-yl)-3-(4-fluorophenyl)-tropane;
(1R,2R,3S)-2-(3-Phenyl-1,2,4-oxadiazol-5-yl)-3-(2-naphthyl)-tropane;
(1R,2R,3S)-3-(3,4-Dichlorophenyl)-tropane-2-aldoxime;
(1R,2R,3S)-3-(3,4-Dichlorophenyl)-tropane-2-O-methyl-aldoxime;
(1R,2R,3S)-3-(3,4-Dichlorophenyl)-tropane-2-O-benzyl-aldoxime;
(1R,2R,3S)-3-(3,4-Dichlorophenyl)-tropane-2-O-ethoxycarbonylmethyl-aldoxime;
(1R,2R,3S)-3-(3,4-Dichlorophenyl)-tropane-2-O-methoxycarbonylmethyl-aldoxime;
(1R,2R,3S)-3-(3,4-Dichlorophenyl)-tropane-2-O-(1-ethoxycarbonyl-1,1-dimethyl-methyl)-aldoxime;
(1R,2R, 3S)-3-(3,4-Dichlorophenyl)-tropane-2-O-carboxymethyl-2-aldoxime;
(1R,2R,3S)-N-Normethyl-3-(3,4-dichlorophenyl)-tropane-2-O-methyl-aldoxime;
(1R,2R,3S)-N-Normethyl-3-(3,4-dichlorophenyl)-tropane-2-O-benzyl-aldoxime;
(1R,2R, 3S)-3-(4-Methylphenyl)-tropane-2-O-methyl-aldoxime;
(1R,2R,3S)-3-(3,4-Dichlorophenyl)-tropane-2-O-(1,1-dimethylethyl)-aldoxime;
(1R,2R,3S)-3-(4-Chlorophenyl)-tropane-2-O-aldoxime;
(1R,2R,3S)-3-(4-Chlorophenyl)-tropane-2-O-methylaldoximehydrochloride;
(1R,2R,3S)-3-(4-Chlorophenyl)-tropane-2-O-methoxycarbonylmethyl-aldoxime;
(1R,2R,3S)-3-(3,4-Dichlorophenyl)-tropane-2-O-(2-propynyl)-aldoxime;
(1R,2R,3S)-3-(3,4-Dichlorophenyl)-tropane-2-O-(2-methylpropyl)-aldoxime;
(1R,2R,3S)-3-(3,4-Dichlorophenyl)-tropane-2-O-cyclopropylmethyl-aldoxime;
(1R,2R,3S)-3-(3,4-Dichlorophenyl)-tropane-2-O-ethyl-aldoxime;
(1R,2R,3S)-2-Methoxymethyl-3-(3,4-dichlorophenyl)-tropane;
(1R,2R,3S)-2-Isopropoxymethyl-3-(3,4-dichlorophenyl)-tropane;
(1R,2R,3S)-2-Ethoxymethyl-3-(3,4-dichlorophenyl)-tropane;
(1R,2R,3S)-2-Ethoxymethyl-3-(3,4-dichlorophenyl)-nortropane;
(1R,2R,3S)-2-Cyclopropylmethyloxymethyl-3-(3,4-dichlorophenyl)-tropane;
(1R,2R,3S)-2-Methoxymethyl-3-(4-chlorophenyl)-tropane;
(1R,2R,3S)-N-Normethyl-2-methoxymethyl-3-(4-chlorophenyl)-tropane;
(1R,2R,3S)-2-Ethoxymethyl-3-(4-chlorophenyl)-tropane;
(1R,2R, 3S)-N-Normethyl-2-methoxymethyl-3-(3,4-dichlorophenyl)-tropane;
(1R,2R,3S)-N-Normethyl-2-ethoxymethyl-3-(3,4-dichlorophenyl)-tropane;
(1R,2R,3S)-N-Normethyl-2-ethoxymethyl-3-(4-chlorophenyl)-tropane;
(1R,2R,3S)-N-Normethyl-2-cyclopropylmethyloxymethyl-3-(4-chlorophenyl)-tropane;
(1R,2R,3S)-2-Cyclopropylmethyloxymethyl-3-(4-chlorophenyl)-tropane;
(1R,2R,3S)-2-Ethylthiomethyl-3-(3,4-dichlorophenyl)-tropane;
(1R,2R,3S)-2-Hydroxymethyl-3-(4-fluorophenyl)-tropane;
(1R,2R,3S)-2-Hydroxymethyl-3-(3,4-dichlorophenyl)-tropane;
(1R,2R, 3S)-N-Normethyl-N-(tert-butoxycarbonyl)-2-hydroxymethyl-3-(3,4-dichlorophenyl)-tropane;
(1R,2R,3S)-2-Hydroxymethyl-3-(4-chlorophenyl)-tropane;
(1R,2R,3S)-2-(3-(2-Furanyl)-1,2,4-oxadiazol-5-yl)-3-(3,4-dichlorophenyl)-tropane;
(1R,2R, 3S)-2-(3-(3-Pyridyl)-1,2,4-oxadiazol-5-yl)-3-(3,4-dichlorophenyl)-tropane;
(1R,2R,3S)-N-Normethyl-N-allyl-2-(3-(4-pyridyl)-1,2,4-oxadiazol-5-yl)-3-(3,4-dichlorophenyl)-tropane;
(1R,2R,3S)-N-Normethyl-N-ethyl-2-(3-(4-pyridyl)-1,2,4-oxadiazol-5-yl)-3-(3,4-dichlorophenyl)-tropane;
(1R,2R,3S)-N-Normethyl-N-(2-hydroxyethyl)-2-(3-(4-pyridyl)-1,2,4-oxadiazol-5-yl)-3-(3,4-dichlorophenyl)-tropane;
(1R,2R,3S)-N-Normethyl-2-(3-(4-pyridyl)-1,2,4-oxadiazol-5-yl)-3-(3,4-dichlorophenyl)-tropane;
(1R,2R,3S)-N-Normethyl-N-allyl-2-(3-(3-pyridyl)-1,2,4-oxadiazol-5-yl)-3-(3,4-dichlorophenyl)-tropane;
(1R,2R,3S)-N-Normethyl-N-allyl-2-(3-(2-pyridyl)-1,2,4-oxadiazol-5-yl)-3-(3,4-dichlorophenyl)-tropane;
(1R,2R,3S)-2-(3-(2-Thienyl)-1,2,4-oxadiazol-5-yl)-3-(4-chlorophenyl)-tropane;
(1R,2R,3S)-2-(3-(2-Thienyl)-1,2,4-oxadiazol-5-yl)-3-(3,4-dichlorophenyl)-tropane;
(1R,2R,3S)-2-(3-(4-Pyridyl)-1,2,4-oxadiazol-5-yl)-3-(3,4-dichlorophenyl)-tropane;
(1R,2R,3S)-2-(3-(2-Pyridyl)-1,2,4-oxadiazol-5-yl)-3-(3,4-dichlorophenyl)-tropane;
(1R,2R,3S)-2-(3-(4-Pyridyl)-1,2,4-oxadiazol-5-yl)-3-(4-chlorophenyl)-tropane;
(1R,2R,3S)-2-(3-(3-Pyridyl)-1,2,4-oxadiazol-5-yl)-3-(4-chlorophenyl)-tropane;
(1R,2R,3S)-2-(3-2-Pyridyl)-1,2,4-oxadiazol-5-yl)-3-(4-chlorophenyl)-tropane;
(1R,2R,3S)-2-(3-Phenyl-1,2,4-oxadiazol-5-yl)-3-(4-fluorophenyl)-tropane;
(1R,2R,3S)-2-(3-Phenyl-1,2,4-oxadiazol-5-yl)-3-(4-methylphenyl)-tropane;
(1R,2R,3S)-2-(3-Benzyl-1,2,4-oxadiazol-5-yl)-3-(4-fluorophenyl)-tropane;
(1R,2R,3S)-2-(3-(4-Phenylphenyl)-1,2,4-oxadiazol-5-yl)-3-(4-fluorophenyl)-tropane;
(1R,2R,3S)-2-(3-Phenyl-1,2,4-oxadiazol-5-yl)-3-(2-naphthyl)-tropane;
(1R,2R,3S)-2-(4-Chlorophenoxy-methyl)-3-(4-fluorophenyl)-tropane;
(1R,2R,3S)-2-(4-Chlorophenoxy-methyl)-3-(4-fluorophenyl)-tropane;
(1R,2R,3S)-2-(4-Chlorophenoxy-methyl)-3-(3,4-dichlorophenyl)-tropane;
(1R,2R,3S)-2-(4-Chlorophenoxy-methyl)-3-(4-methylphenyl)-tropane;
(1R,2R,3S)-2-(4-Benzoyloxy-methyl)-3-(4-fluorophenyl)-tropane;
(1R,2R,3S)-2-Carbomethoxy-3-(2-naphthyl)-tropane;
(1R,2R,3S)-2-Carbomethoxy-3-(3,4-dichlorophenyl)-tropane;
(1R,2R, 3S)-2-Carbomethoxy-3-benzyl-tropane;
(1R,2R,3S)-2-Carbomethoxy-3-(4-chlorophenyl)-tropane;
(1R,2R,3S)-2-Carbomethoxy-3-(4-methylphenyl)-tropane;
(1R,2R,3S)-2-Carbomethoxy-3-(1-naphthyl)-tropane;
(1R,2R,3S)-2-Carbomethoxy-3-(4-phenylphenyl)-tropane;
(1R,2R,3S)-2-Carbomethoxy-3-(4-t-butyl-phenyl)-tropane;
(1R,2R,3S)-2-(4-Fluoro-benzoyl)-3-(4-fluorophenyl)-tropane; or
   a pharmaceutically acceptable addition salt thereof.

Most preferred is the compound of formula (IA) 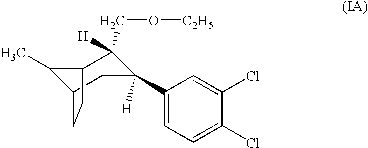
or a pharmaceutically acceptable salt thereof, in particular the citrate thereof.

Dopamine agonists which may be used include any which are known to the skilled person and those which will become available in the future. Examples include: amisulpride, amisulpride, bromocriptine, buspirone, cabergoline, docarpamine, dopexamine, etilevodopa, fenoldopam, ibopamine, lisuride, nolomirole, pergolide, piripedil, pramipexole, quinagolide, quinelorane, ropinirole, rotigotine, roxindole, sibenadet, sumanirole, talipexole and terguride.

Preferred is a combination of the compound of formula (IA) with a dopamine agonists selected from the group consisting of: pramipexole, pramipexole dihydrochloride, pramipexole dihydrobromide, ropinirole, rotigotine, roxindole, sibenadet, and talipexole.

Most preferred is a combination of the compound of formula (IA) with pramipexole, which is (S)-2-amino-4,5,6,7-tetrahydro-6-(propylamino)benzothiazole of formula (II) 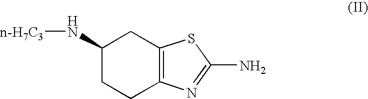
the dihydrochloride thereof or the dihydrochloride monohydrate thereof.

The pharmaceutical compositions of the present invention are suitable for oral, intravenous, intravascular, intraperitoneal, subcutaneous, intramuscular, inhalative, topical, patch, or suppository administration.

The pharmaceutical compositions of the present invention are preferably in unit dosage forms, such as tablets, pills, capsules, powders, granules, sterile parenteral solutions, or suspensions, metered aerosol or liquid sprays, drops, ampoules, transdermal patches, auto-injector devices, or suppositories; for oral, parenteral, intranasal, sublingual, or rectal administration, or for administration by inhalation or insufflation. For preparing solid compositions, such as tablets, the principal active ingredient is mixed with a pharmaceutical carrier, e.g., conventional tabletting ingredients, such as corn starch, cellulose, carboxymethylcellulose, hydroxypropylmethylcellulose, lactose, sucrose, sorbitol, talc, silicon dioxide, polyethylene glycol, stearic acid, magnesium stearate, and dicalcium phosphate, or gums, or surfactants, such as sorbitan monooleate, polyethylene glycol, and other pharmaceutical diluents, e.g., water, to form a solid pre-formulation composition containing a homogeneous mixture of a compound of the present invention, or a pharmaceutically acceptable salt thereof. When referring to these pre-formulation compositions as homogeneous, it is meant that the active ingredient is dispersed evenly throughout the composition so that the composition may be readily subdivided into equally effective unit dosage forms such as tablets, pills, and capsules.

This solid pre-formulation composition is then subdivided into unit dosage forms of the type described above containing from 0.01 to 10,000 mg, in particular 0.05 to about 500 mg, most preferably 0.75 to 250 mg of each active ingredient of the present invention. Typical unit dosage forms contain from 1 to 100 mg, for example 1, 2, 5, 10, 25, 50, or 100 mg, of each active ingredient.

Most preferably, 0.025 to 1.5 mg, in particular 0.044, 0.088, 0.18, 0.35, 0.7, or 1.1 mg, of pramipexole are adminstered together with 0.05 to 1.5 mg, in particular 0.06, 0.125, 0.25, 0.5, or 1.0 mg, of the compound of formula IA.

The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Similarly, cachets and lozenges are included. Tablets, powders, capsules, pills, cachets, and lozenges can be used as solid forms suitable for oral administration.

The liquid forms in which the novel compositions of the present invention may be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles. Suitable dispersing or suspending agents for aqueous suspensions include synthetic and natural gums, such as tragacanth, acacia, alginate, dextran, sodium carboxymethylcellulose, methylcellulose, polyvinyl-pyrrolidone, or gelatin.

For preparing suppositories, a low melting wax, such as admixture of fatty acid glycerides or cocoa butter, is first melted and the active component is dispersed homogeneously therein, as by stirring. The molten homogenous mixture is then poured into convenient sized molds, allowed to cool, and thereby to solidify.

Formulations suitable for vaginal administration may be presented as pessaries, tampons, creams, gels, pastes, foams, or sprays containing, in addition to the active ingredient, such carriers as are known in the art to be appropriate.

Administration to the respiratory tract may also be achieved by means of an aerosol formulation in which the active ingredient is provided in a pressurized pack with a suitable propellant, such as a chlorofluorocarbon (CFC) or fluorohydrocarbon (HFC) for example dichlorodifluoromethane, trichlorofluoromethane, dichlorotetrafluoroethane, 1,1,1,2-tetrafluoroethan (HFC-134(a)), or 1,1,1,2,3,3,3-heptafluoroprpane, carbon dioxide, or other suitable gas. The aerosol may conveniently also contain a surfactant, such as lecithin, and/or a co-solvent, such as ethanol. The dose of drug may be controlled by provision of a metered valve.

Alternatively the active ingredients may be provided in the form of a dry powder, for example, a powder mix of the compound in a suitable powder base such as lactose, starch, starch derivatives, such as hydroxypropylmethyl cellulose and polyvinylpyrrolidone (PVP). Conveniently the powder carrier will form a gel in the nasal cavity. The powder composition may be presented in unit dose form, for example, in capsules or cartridges of, e.g., gelatin, or blister packs from which the powder may be administered by means of an inhaler.

In formulations intended for administration to the respiratory tract, including intranasal formulations, the compound will generally have a small particle size, for example, of the order of 5 microns or less. Such a particle size may be obtained by means known in the art, for example, by micronization.

In a preferred embodiment of the inventive kit of parts pramipexole is administered via a transdermal patch as disclosed for example by EP 0 428 038 Case 3/0327 and the compound of formula (IA) is administered orally.

For the treatment of a Parkinson's Disease or depression, a suitable dosage level is about 0.01 to 1.0 mg/kg per day, preferably about 0.02 to 0.5 mg/kg per day, and especially about 0.05 to 0.2 mg/kg of body weight per day of each active ingredient. The compounds may be administered on a regimen of 1 to 4 times per day. In some cases, however, dosage outside these limits may be used.

Most preferably, the composition of the invention will be used for the treatment or prevention of one or more of the following neurodegenerative conditions: Parkinson's Disease, pseudodementia, dementia, dementia of Alzheimer-type, Alzheimer's Disease, presenile dementia, senile dementia, Lewy-Body-dementia, Down syndrome, fronto temporal dementia, HIV related dementia, Pick's Disease, cerebrovascular dementia, multi-infarct dementia, memory deficits, attention deficits, cognitive dysfunction, memory dysfunction, mild cognitive impairment, age associated memory impairment, ageing-associated cognitive decline, age-related cognitive decline, ALS, and multiple system atrophy.

Preferably the weight ratio of the monoamine neurotransmitter re-uptake inhibitor to the dopamine agonist ranges from 50:1 to 1:300, in particular from 1:1 to 1:200, and most preferably from 1:2 to 1:50.

The Examples that follow serve to illustrate some formulations according to the invention. They are intended solely as possible procedures described by way of example, without restricting the invention to their content.

### EXAMPLE 1

A pharmaceutical composition is prepared by combining pramipexole, in either its racemic or entantiomeric form, with the compound of formula (IA) in a pharmaceutically acceptable carrier. The composition contains respective amounts of pramipexole and formula (IA) to deliver on a daily basis between about 0.05 mg to about 1.5 mg pramipexole and between about 0.1 mg to about 2 mg of formula (IA) per kilogram of patient body weight (for example, 6 mg to 120 mg formula (IA) for a person weighing 60 kg). The composition is administered to a patient for the treatment of Parkinsonism, Alzheimer's Disease or depression.

### EXAMPLE 2

A first pharmaceutical composition is prepared by combining pramipexole, in either its racemic or enantiomeric form, in a pharmaceutically acceptable carrier such that it can deliver between about 0.05 mg to about 1.5 mg pramipexole on a daily basis.

A second pharmaceutical composition is prepared by combining formula (IA) in a pharmaceutically acceptable carrier such that it can deliver between about 0.05 mg to about 2 mg of formula (IA) per kilogram of patient body weight on a daily basis.

The first composition is administered to a patient suffering from Parkinsonism, Alzheimer's Disease or depression once, twice, three times, four times or six times daily such that the daily dosage is between about 0.1 to about 10 mg. The second composition is administered to the same patient at the same time as the administration of the first composition or any time within 24 hours of the administration of the first composition once, twice, three times, four times or six times daily such that the daily dosage is between about 0.1 mg to about 2 mg of formula (IA) per kilogram of patient body weight.

Alternatively, the second composition could first be administered, followed by the administration of the first composition as disclosed at the same time, or within 24 hours thereof.

### EXAMPLE 3

### Film-Coated Tablet

| | |
|---|---|
| Constituents | mg/tablet |

Core

| | |
|---|---|
| (IA) citrate | 0.396 |
| Pramipexole dihydrochloride | 0.240 |
| Lactose monohydrate (200 mesh) | 101.130 |
| Microcrystalline cellulose (grade PH 101) | 69.000 |
| Corn starch | 6.300 |
| Purified water* | (q.s.)* |
| Sodiumstarchglycolate | 3.600 |
| Colloidal silicon dioxide | 0.900 |
| Magnesium stearate | 1.800 |

Coating

| | |
|---|---|
| Hydroxyproylmethylcellulose 2910 | 2.750 |
| Polyethylene Glycol 400 | 0.325 |
| Titanium dioxide | 1.000 |
| Talc | 0.925 |
| Purified water* | (q.s.)* |
| Total weight film coated tablet | 185.000 |

*does not appear in final product

### EXAMPLE 4

### Encapsulated Granules

| | |
|---|---|
| Constituents | mg/capsule |

Granules

| | |
|---|---|
| (IA) citrate | 0.238 |
| Pramipexole dihydrochloride | 0.801 |
| Microcrystalline cellulose | 71.592 |
| Dibasic calcium phosphate, anhydrous | 71.494 |
| Hypromellose | 2.750 |
| Carboxymethylcellulose sodium, crosslinked | 2.000 |
| Purified water* | (q.s.)* |
| Colloidal silicon dioxide | 0.375 |
| Magnesium stearate | 0.750 |

Capsules

| | |
|---|---|
| Granules | 150.000 |
| Hard-gelatin capsule (size 2) | 61.000 |
| Total weight capsule | 211.000 |

*does not appear in final product

### EXAMPLE 5

### Bilayer Tablet

| | Mg/tablet |
|---|---|
| 1^{st} Tablet Layer Constituents | |
| (IA) citrate | 0.396 |
| Lactose monohydrate (200 mesh) | 70.104 |
| Microcrystalline cellulose (grade PH 101) | 42.000 |
| Corn starch | 4.200 |
| Purified water* | (q.s.)* |
| Sodiumstarchglycolate | 2.400 |
| Magnesium stearate | 0.900 |
| 2^{nd} Tablet Layer Constituents | |
| Pramipexole dihydrochloride | 5.342 |
| Sorbitol, powder | 120.308 |
| Microcrystalline Cellulose | 14.000 |
| Crospovidone | 2.800 |
| Magnesium stearate | 1.750 |
| Total weight bilayer tablet | 260.000 |

*does not appear in final product

1. A composition comprising:
a 2,3-disubstituted tropane moiety, or a tautomer, pharmaceutically acceptable salt, solvate, or physiologically functional derivative thereof,
at least one dopamine agonist, or a pharmaceutically acceptable salt, solvate, or physiologically functional derivative thereof.

2. A composition according to claim 1, wherein the 2,3-disubstituted tropane moiety is a compound of formula (I) 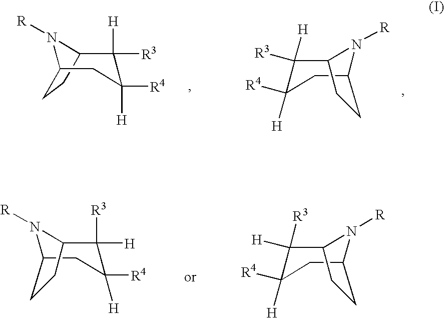
or an addition salt or N-oxide thereof, wherein
R is hydrogen, alkyl, alkenyl, alkynyl, cycloalkyl, cycloalkylalkyl, or 2-hydroxyethyl;
R³ is
CH₂—X—R′, wherein
X is O, S, or NR″, wherein
R″ is hydrogen or alkyl; and
R′ is
alkyl, alkenyl, alkynyl, cycloalkyl, cycloalkylalkyl, or —CO-alkyl;
heteroaryl, which may be substituted one or more times with alkyl, cycloalkyl, or cycloalkylalkyl;
phenyl, which may be substituted one or more times with substituents selected from the group consisting of halogen, CF₃, CN, alkoxy, alkyl, alkenyl, alkynyl, amino, nitro, and heteroaryl;
phenylphenyl;
pyridyl, which may be substituted one or more times with substituents selected from the group consisting of halogen, CF₃, CN, alkoxy, alkyl, alkenyl, alkynyl, amino, nitro, and heteroaryl;
thienyl, which may be substituted one or more times with substituents selected from the group consisting of halogen, CF₃, CN, alkoxy, alkyl, alkenyl, alkynyl, amino, nitro, and heteroaryl; or
benzyl, which may be substituted one or more times with substituents selected from the group consisting of halogen, CF₃, CN, alkoxy, alkyl, alkenyl, alkynyl, amino, nitro, and heteroaryl; or
(CH₂)ₙCO₂R¹¹, COR¹¹″, or CH₂R¹² wherein
R¹¹ is
alkyl, cycloalkyl, or cycloalkylalkyl;
phenyl, which may be substituted one or more times with substituents selected from the group consisting of halogen, CF₃, CN, alkoxy, alkyl, alkenyl, alkynyl, amino, nitro, and heteroaryl;
phenylphenyl;
pyridyl, which may be substituted one or more times with substituents selected from the group consisting of halogen, CF₃, CN, alkoxy, alkyl, alkenyl, alkynyl, amino, nitro, and heteroaryl;
thienyl or O-thienyl, which may be substituted one or more times with substituents selected from the group consisting of halogen, CF₃, CN, alkoxy, alkyl, alkenyl, alkynyl, amino, nitro, and heteroaryl; or
benzyl;
n is 0 or 1; and
R¹² is
O-phenyl, which may be substituted one or more times with substituents selected from the group consisting of halogen, CF₃, CN, alkoxy, alkyl, alkenyl, alkynyl, amino, nitro, and heteroaryl; or
O—CO-phenyl, which may be substituted one or more times with substituents selected from the group consisting of halogen, CF₃, CN, alkoxy, alkyl, alkenyl, alkynyl, amino, nitro, and heteroaryl; or
CH═NOR′ wherein
R′ is
hydrogen or O-hydrogen;
alkyl, O-alkyl, cycloalkyl, cycloalkylalkyl, alkenyl, alkynyl or aryl, all of which may be substituted with —COOH;
—COO-alkyl;
—COO-cycloalkyl; or
phenyl, which may be substituted one or more times with substituents selected from the group consisting of halogen, CF₃, CN, alkyl, cycloalkyl, alkoxy, cycloalkoxy, alkenyl, alkynyl, amino, and nitro; and
R⁴ is
3,4-methylenedioxyphenyl; or
phenyl, benzyl, naphthyl, or heteroaryl, all of which may be substituted one or more times with substituents selected from the group consisting of halogen, CF3, CN, alkoxy, cycloalkoxy, alkyl, cycloalkyl, alkenyl, alkynyl, amino, nitro, and heteroaryl.

3. A composition according to claim 2, wherein R⁴ is phenyl, which is substituted once or twice with substituents selected from the group consisting of: halogen, CF₃, CN, alkoxy, cycloalkoxy, alkyl, cycloalkyl, alkenyl, alkynyl, amino, nitro, and heteroaryl.

4. A composition according to claim 2, wherein R⁴ is phenyl, which is substituted once or twice with chlorine.

5. A composition according to claim 1, wherein the 2,3-disubstituted tropane moiety is a compound of formula (I) 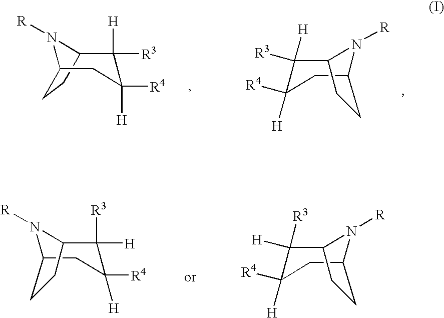
or an addition salt or N-oxide thereof, wherein
R is hydrogen, alkyl, alkenyl, alkynyl, cycloalkyl, cycloalkylalkyl, or 2-hydroxyethyl;
R³ is
CH₂—X—R′, wherein
X is O, S, or NR″, wherein
R″ is hydrogen or alkyl; and
R′ is alkyl, alkenyl, alkynyl, cycloalkyl, cycloalkylalkyl, or —CO-alkyl; and
R⁴ is
3,4-methylenedioxyphenyl; or
phenyl, benzyl, naphthyl, or heteroaryl, all of which may be substituted one or more times with substituents selected from the group consisting of halogen, CF3, CN, alkoxy, cycloalkoxy, alkyl, cycloalkyl, alkenyl, alkynyl, amino, nitro, and heteroaryl.

6. A composition according to claim 1, wherein the 2,3-disubstituted tropane moiety is a compound of formula (I) 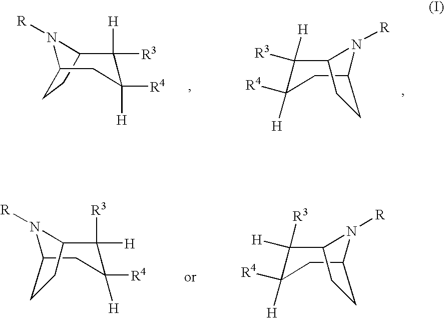
or an addition salt or N-oxide thereof, wherein
R is hydrogen, alkyl, alkenyl, alkynyl, cycloalkyl, cycloalkylalkyl, or 2-hydroxyethyl;
R³ is
CH═NOR′ wherein
R′ is
hydrogen;
alkyl, cycloalkyl, cycloalkylalkyl, alkenyl, alkynyl or aryl, all of which may be substituted with —COOH;
—COO-alkyl;
—COO-cycloalkyl; or
phenyl, which may be substituted one or more times with substituents selected from the group consisting of halogen, CF₃, CN, alkyl, cycloalkyl, alkoxy, cycloalkoxy, alkenyl, alkynyl, amino, and nitro; and
R⁴ is
3,4-methylenedioxyphenyl; or
phenyl, benzyl, naphthyl, or heteroaryl, all of which may be substituted one or more times with substituents selected from the group consisting of halogen, CF3, CN, alkoxy, cycloalkoxy, alkyl, cycloalkyl, alkenyl, alkynyl, amino, nitro, and heteroaryl.

7. A composition according to claim 1, wherein the 2,3-disubstituted tropane moiety is a compound of formula (I1) 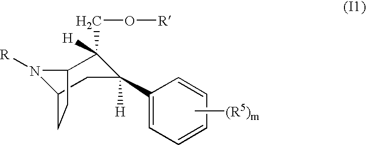
wherein
R represents a hydrogen atom or a C₁₋₆ alkyl group;
R⁵ represents a halogen atom or a CF₃ or cyano group;
R′ represents a hydrogen atom or a C₁₋₆ alkyl, or C₃₋₆-cycloalkyl-C₁₋₃-alkyl group; and
m is 0 or an integer from 1 to 3;
or a tautomer, pharmaceutically acceptable salt, solvate, or physiologically functional derivative thereof.

8. A composition according to claim 7, wherein:
R represents hydrogen, or a methyl or ethyl group;
R⁵ represents fluorine, chlorine, or bromine;
R′ represents a methyl, ethyl, or n-propyl group; and
m is 1 or 2.

9. A composition according to claim 1, wherein the 2,3-disubstituted tropane moiety is selected from the group consisting of:
(1R,2R, 3S)-2-(3-Cyclopropyl-1,2,4-oxadiazol-5-yl)-3-(4-fluorophenyl)-tropane;
(1R,2R,3S)-2-(3-Phenyl-1,2,4-oxadiazol-5-yl)-3-(4-fluorophenyl)-tropane;
(1R,2R,3S)-2-(3-Phenyl-1,2,4-oxadiazol-5-yl)-3-(4-methylphenyl)-tropane;
(1R,2R,3S)-2-(3-Benyl-1,2,4-oxadiazol-5-yl)-3-(4-fluorophenyl)-tropane;
(1R,2R,3S)-2-(3-(4-Phenyl-phenyl)-1,2,4-oxadiazol-5-yl)-3-(4-fluorophenyl)-tropane;
(1R,2R,3S)-2-(3-Phenyl-1,2,4-oxadiazol-5-yl)-3-(2-naphthyl)-tropane;
(1R,2R,3S)-3-(3,4-Dichlorophenyl)-tropane-2-aldoxime;
(1R,2R,3S)-3-(3,4-Dichlorophenyl)-tropane-2-O-methyl-aldoxime;
(1R,2R,3S)-3-(3,4-Dichlorophenyl)-tropane-2-O-benzyl-aldoxime;
(1R,2R, 3S)-3-(3,4-Dichlorophenyl)-tropane-2-O-ethoxycarbonylmethyl-aldoxime;
(1R,2R,3S)-3-(3,4-Dichlorophenyl)-tropane-2-O-methoxycarbonylmethyl-aldoxime;
(1R,2R,3S)-3-(3,4-Dichlorophenyl)-tropane-2-O-(1-ethoxycarbonyl-1,1-dimethyl-methyl)-aldoxime;
(1R,2R,3S)-3-(3,4-Dichlorophenyl)-tropane-2-O-carboxymethyl-2-aldoxime;
(1R,2R,3S)-N-Normethyl-3-(3,4-dichlorophenyl)-tropane-2-O-methyl-aldoxime;
(1R,2R,3S)-N-Normethyl-3-(3,4-dichlorophenyl)-tropane-2-O-benzyl-aldoxime;
(1R,2R,3S)-3-(4-Methylphenyl)-tropane-2-O-methyl-aldoxime;
(1R,2R,3S)-3-(3,4-Dichlorophenyl)-tropane-2-O-(1,1-dimethylethyl)-aldoxime;
(1R,2R,3S)-3-(4-Chlorophenyl)-tropane-2-O-aldoxime;
(1R,2R,3S)-3-(4-Chlorophenyl)-tropane-2-O-methylaldoxime hydrochloride;
(1R,2R,3S)-3-(4-Chlorophenyl)-tropane-2-O-methoxycarbonylmethyl-aldoxime;
(1R,2R,3S)-3-(3,4-Dichlorophenyl)-tropane-2-O-(2-propynyl)-aldoxime;
(1R,2R,3S)-3-(3,4-Dichlorophenyl)-tropane-2-O-(2-methylpropyl)-aldoxime;
(1R,2R,3S)-3-(3,4-Dichlorophenyl)-tropane-2-O-cyclopropylmethyl-aldoxime;
(1R,2R,3S)-3-(3,4-Dichlorophenyl)-tropane-2-O-ethyl-aldoxime;
(1R,2R,3S)-2-Methoxymethyl-3-(3,4-dichlorophenyl)-tropane;
(1R,2R,3S)-2-Isopropoxymethyl-3-(3,4-dichlorophenyl)-tropane;
(1R,2R,3S)-2-Ethoxymethyl-3-(3,4-dichlorophenyl)-tropane;
(1R,2R,3S)-2-Ethoxymethyl-3-(3,4-dichlorophenyl)-nortropane;
(1R,2R, 3S)-2-Cyclopropylmethyloxymethyl-3-(3,4-dichlorophenyl)-tropane;
(1R,2R,3S)-2-Methoxymethyl-3-(4-chlorophenyl)-tropane;
(1R,2R,3S)-N-Normethyl-2-methoxymethyl-3-(4-chlorophenyl)-tropane;
(1R,2R,3S)-2-Ethoxymethyl-3-(4-chlorophenyl)-tropane;
(1R,2R,3S)-N-Normethyl-2-methoxymethyl-3-(3,4-dichlorophenyl)-tropane;
(1R,2R,3S)-N-Normethyl-2-ethoxymethyl-3-(3,4-dichlorophenyl)-tropane;
(1R,2R,3S)-N-Normethyl-2-ethoxymethyl-3-(4-chlorophenyl)-tropane;
(1R,2R,3S)-N-Normethyl-2-cyclopropylmethyloxymethyl-3-(4-chlorophenyl)-tropane;
(1R,2R,3S)-2-Cyclopropylmethyloxymethyl-3-(4-chlorophenyl)-tropane;
(1R,2R,3S)-2-Ethylthiomethyl-3-(3,4-dichlorophenyl)-tropane;
(1R,2R,3S)-2-Hydroxymethyl-3-(4-fluorophenyl)-tropane;
(1R,2R,3S)-2-Hydroxymethyl-3-(3,4-dichlorophenyl)-tropane;
(1R,2R,3S)-N-Normethyl-N-(tert-butoxycarbonyl)-2-hydroxymethyl-3-(3,4-dichlorophenyl)-tropane;
(1R,2R,3S)-2-Hydroxymethyl-3-(4-chlorophenyl)-tropane;
(1R,2R, 3S)-2-(3-(2-Furanyl)-1,2,4-oxadiazol-5-yl)-3-(3,4-dichlorophenyl)-tropane;
(1R,2R,3S)-2-(3-(3-Pyridyl)-1,2,4-oxadiazol-5-yl)-3-(3,4-dichlorophenyl)-tropane;
(1R,2R, 3S)-N-Normethyl-N-allyl-2-(3-(4-pyridyl)-1,2,4-oxadiazol-5-yl)-3-(3,4-dichlorophenyl)-tropane;
(1R,2R,3S)-N-Normethyl-N-ethyl-2-(3-(4-pyridyl)-1,2,4-oxadiazol-5-yl)-3-(3,4-dichlorophenyl)-tropane;
(1R,2R,3S)-N-Normethyl-N-(2-hydroxyethyl)-2-(3-(4-pyridyl)-1,2,4-oxadiazol-5-yl)-3-(3,4-dichlorophenyl)-tropane;
(1R,2R,3S)-N-Normethyl-2-(3-(4-pyridyl)-1,2,4-oxadiazol-5-yl)-3-(3,4-dichlorophenyl)-tropane;
(1R,2R,3S)-N-Normethyl-N-allyl-2-(3-(3-pyridyl)-1,2,4-oxadiazol-5-yl)-3-(3,4-dichlorophenyl)-tropane;
(1R,2R,3S)-N-Normethyl-N-allyl-2-(3-(2-pyridyl)-1,2,4-oxadiazol-5-yl)-3-(3,4-dichlorophenyl)-tropane;
(1R,2R,3S)-2-(3-(2-Thienyl)-1,2,4-oxadiazol-5-yl)-3-(4-chlorophenyl)-tropane;
(1R,2R,3S)-2-(3-(2-Thienyl)-1,2,4-oxadiazol-5-yl)-3-(3,4-dichlorophenyl)-tropane;
(1R,2R,3S)-2-(3-(4-Pyridyl)-1,2,4-oxadiazol-5-yl)-3-(3,4-dichlorophenyl)-tropane;
(1R,2R,3S)-2-(3-(2-Pyridyl)-1,2,4-oxadiazol-5-yl)-3-(3,4-dichlorophenyl)-tropane;
(1R,2R,3S)-2-(3-(4-Pyridyl)-1,2,4-oxadiazol-5-yl)-3-(4-chlorophenyl)-tropane;
(1R,2R, 3S)-2-(3-(3-Pyridyl)-1,2,4-oxadiazol-5-yl)-3-(4-chlorophenyl)-tropane;
(1R,2R,3S)-2-(3-2-Pyridyl)-1,2,4-oxadiazol-5-yl)-3-(4-chlorophenyl)-tropane;
(1R,2R,3S)-2-(3-Phenyl-1,2,4-oxadiazol-5-yl)-3-(4-fluorophenyl)-tropane;
(1R,2R,3S)-2-(3-Phenyl-1,2,4-oxadiazol-5-yl)-3-(4-methylphenyl)-tropane;
(1R,2R,3S)-2-(3-Benzyl-1,2,4-oxadiazol-5-yl)-3-(4-fluorophenyl)-tropane;
(1R,2R,3S)-2-(3-(4-Phenylphenyl)-1,2,4-oxadiazol-5-yl)-3-(4-fluorophenyl)-tropane;
(1R,2R,3S)-2-(3-Phenyl-1,2,4-oxadiazol-5-yl)-3-(2-naphthyl)-tropane;
(1R,2R,3S)-2-(4-Chlorophenoxy-methyl)-3-(4-fluorophenyl)-tropane;
(1R,2R,3S)-2-(4-Chlorophenoxy-methyl)-3-(4-fluorophenyl)-tropane;
(1R,2R,3S)-2-(4-Chlorophenoxy-methyl)-3-(3,4-dichlorophenyl)-tropane;
(1R,2R,3S)-2-(4-Chlorophenoxy-methyl)-3-(4-methylphenyl)-tropane;
(1R,2R,3S)-2-(4-Benzoyloxy-methyl)-3-(4-fluorophenyl)-tropane;
(1R,2R,3S)-2-Carbomethoxy-3-(2-naphthyl)-tropane;
(1R,2R, 3S)-2-Carbomethoxy-3-(3,4-dichlorophenyl)-tropane;
(1R,2R,3S)-2-Carbomethoxy-3-benzyl-tropane;
(1R,2R, 3S)-2-Carbomethoxy-3-(4-chlorophenyl)-tropane;
(1R,2R,3S)-2-Carbomethoxy-3-(4-methylphenyl)-tropane;
(1R,2R,3S)-2-Carbomethoxy-3-(1-naphthyl)-tropane;
(1R,2R,3S)-2-Carbomethoxy-3-(4-phenylphenyl)-tropane;
(1R,2R,3S)-2-Carbomethoxy-3-(4-t-butyl-phenyl)-tropane; and
(1R,2R,3S)-2-(4-Fluoro-benzoyl)-3-(4-fluorophenyl)-tropane,
or a pharmaceutically acceptable addition salt of such 2,3-disubstituted tropane moiety.

10. A composition according to claim 1, wherein the 2,3-disubstituted tropane moiety is a compound of formula (IA) 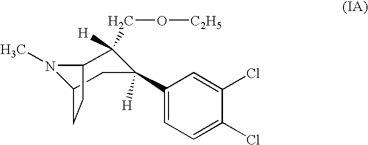
or a pharmaceutically acceptable salt thereof;

11. A composition according to claim 1, wherein the dopamine agonist is selected from the group consisting of: amisulpride, amisulpride, bromocriptine, buspirone, cabergoline, docarpamine, dopexamine, etilevodopa, fenoldopam, ibopamine, lisuride, nolomirole, pergolide, piripedil, pramipexole, quinagolide, quinelorane, ropinirole, rotigotine, roxindole, sibenadet, sumanirole, talipexole and terguride, and mixtures and combinations thereof.

12. A composition according to claim 1 that is suitable for oral, intravenous, intravascular, intraperitoneal, subcutaneous, intramuscular, inhalative, topical, patch, or suppository administration.

13. A composition according to claim 1, wherein the 2,3-disubstituted tropane moiety and the dopamine agonist are each present in a weight of about 0.05 mg to about 10,000 mg.

14. A composition according to claim 1, wherein the weight ratio of the 2,3-disubstituted tropane moiety to the dopamine agonist is about 50:1 to about 1:300.

15. A method for the prevention or treatment of a disease or disorder that is responsive to a monoamine neurotransmitter re-uptake inhibitor, dopamine agonism, or both, the method comprising jointly, separately, or sequentially administering, to a patient in need thereof, effective amounts of: (i) a 2,3-disubstituted tropane moiety, or a tautomer, pharmaceutically acceptable salt, solvate, or physiologically functional derivative thereof, and (ii) an dopamine agonist, or a pharmaceutically acceptable salt, solvate, or physiologically functional derivative thereof.

16. A method according to claim 15, wherein said disease or disorder is selected from the group consisting of: pseudodementia, dementia, Alzheimer-type dementia, Alzheimer's Disease, presenile dementia, senile dementia, Lewy-Body dementia, Down syndrome, fronto-temporal dementia, HIV-related dementia, Pick's Disease, Parkinson's Disease, cerebrovascular dementia, multi-infarct dementia, memory deficits, attention deficits, cognitive dysfunction, memory dysfunction, mild cognitive impairment (MCI), age-associated memory impairment (AAMI), ageing-associated cognitive decline, age-related cognitive decline, and multiple system atrophy.

17. A method according to claim 15, wherein the effective amounts of the 2,3-disubstituted tropane moiety and the dopamine agonist are each about 0.5 mg to about 10,000 mg per day.

18. A method according to claim 15, wherein the weight ratio of the effective amount of the 2,3-disubstituted tropane moiety to the effective amount of the dopamine agonist is about 50:1 to about 1:300.

19. A pharmaceutical kit comprising comprising:
a first dosage form comprising a monoamine neurotransmitter re-uptake inhibitor comprising a 2,3-disubstituted tropane moiety, or a tautomer, pharmaceutically acceptable salt, solvate, or physiologically functional derivative thereof; and
a second dosage form comprising a dopamine agonist, or a pharmaceutically acceptable salt, solvate, or physiologically functional derivative thereof.

20. A pharmaceutical kit according to claim 19, wherein the first dosage form comprises a compound of formula (IA): 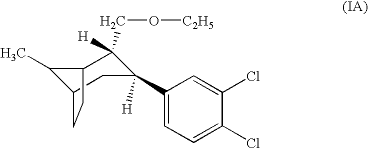
and the second dosage form comprises a compound selected from the group consisting of: amisulpride, amisulpride, bromocriptine, buspirone, cabergoline, docarpamine, dopexamine, etilevodopa, fenoldopam, ibopamine, lisuride, nolomirole, pergolide, piripedil, pramipexole, quinagolide, quinelorane, ropinirole, rotigotine, roxindole, sibenadet, sumanirole, talipexole and terguride, and mixtures and combinations thereof.
